(19) United States
(12) Patent Application Publication
(10) Pub. No.: US 2008/0319447 A1
(43) Pub. Date: Dec. 25, 2008

(54) Device for the Application of Resilient Material Between Vertebras

(76) Inventors: Morgan Andersson, Lit (SE); Matts Andersson, Lerum (SE)

Correspondence Address: YOUNG & THOMPSON, 209 Madison Street, Suite 500, ALEXANDRIA, VA, 22314, US

(21) Appl. No.: 11/997,908

(22) PCT Filed: Jul. 27, 2006
(86) PCT No.: PCT/SE2006/000915

§ 371 Date: Jul. 9, 2008

(30) Foreign Application Priority Data
Aug. 5, 2005 (SE) 0501784-3

(51) Int. Cl. A61B 17/58

(52) U.S. Cl. 606/99; 606/90

(57) ABSTRACT

A device is used for inserting resilient material between the vertebras in a spinal column. The device incorporates first and second units which support portions arranged to cause a separating action upon the vertebras via surfaces present thereon. The separating actuation by means of the portions can be effected by means of an actuating member at or on one or both of the units or portions respectively. Insertion of the resilient material is arranged to take place at a separation height exceeding the height of the material. In such a manner is obtained a tool which is efficiently operating and comparatively technically simple for separating vertebras.

The present invention is related to a device for application of resilient material between vertebras.

It is earlier known to attend to wear at vertebra membranes between adjacent positioned vertebras by means of arthrodesis. It has also been suggested that resilient material shall be applied between the pair of vertebra in question in order to replace the natural, worn out membrane, see i.a. PCT/SE02/02383 filed by one of the inventors of the present patent application.

In connection to material application there is a requirement for a reliable and well operating tool, which enables a clear-cut and rapid application of the material. The aim of the present invention is i.a. to solve this problem.

There is also a requirement, that the tool shall not gather contaminations, but that it can be efficiently cleaned after every occasion of use. There is also a need that the tool can use the properties of the human body, and e.g. utilize the spring back properties which are present in the muscles around a vertebral column. The invention solves also this complex of problems.

The things which mainly can be considered to be characterizing for a device according to the invention is that it incorporates first and second units, which has portions adapted to exert separating action on the vertebras via surfaces present thereon, that the separating action by means of these portions can be executed by means of an actuating member at or on one or both of the units or portions, respectively and that the application of the resilient material is arranged to take place at a degree of separation exceeding the height of the material.

In a further development of the inventive idea the device incorporates a third unit, which is displaceably supported in the longitudinal direction in relation to the first and the second units. The actuating member may in response to actuating motions cause the longitudinal displacement of the third unit in relation to the first and second units. At longitudinal displacement of the third unit away from the portions, the third unit might be arranged to cause separating motion between the first and second units and thereby effect separating movements between the portions. The third unit can, at its inner side, carry a flange which can cooperate with the first unit for effecting the separating movement between the first and second units when the third unit is displaced in relation to the first and the second units by means of the actuating member. The portions might also be situated at the first ends of the first and second units and the actuating member can be arranged at the second ends of the first and second units. In one embodiment the first and second units are pivotably and detachably supported at the second end of the first and second units. The actuating member can incorporate a screw, which is supported at its end in a threaded recess in the third unit and a manually operable actuating means, e.g. a handle, which in one embodiment is adapted in dependency of a turning movement to cause the longitudinal displacement of the third unit in relation to the second unit. In further embodiments of the inventive idea the first and second units are arranged to form an internal channel, via which the resilient material is insertable between the separated vertebras. The device might via the portions be insertable via a passage provided the surface of the human body ant the vertebras by means of a member arranged to expand and maintain a surgical operation in an expanded condition. The last mentioned member might incorporate a spiral, e.g. made of plastic material, and a cloth positioned outside the spiral, adapted to prevent penetration of solid body product residuals into the passage. The member or the spiral with cloth can have substantially the shape of a cone or a frustrum of a cone with the smaller part facing the vertebras. The first, second and third unit can furthermore be dismountable for making cleaning possible.

By means of what has been suggested here above, outstanding advantages are obtained by means of a clear-cut and simplified application of the resilient material. A surgical operation can be effected in a known manner and a passage for a tool or an instrument can be established. The tool can be provided for simple handling and can be applied via the member expanding and maintaining the surgical operation. By means of the tool the vertebras are separated to a distance where the resilient material can be inserted, whereupon the vertebras, when the tool is removed, can spring back by aid of the muscles of the vertebral column and come into cooperation with the material.

An embodiment of a device suggested at present and having the characteristics significant for the invention shall hereinafter be described with reference to the accompanying drawings, where

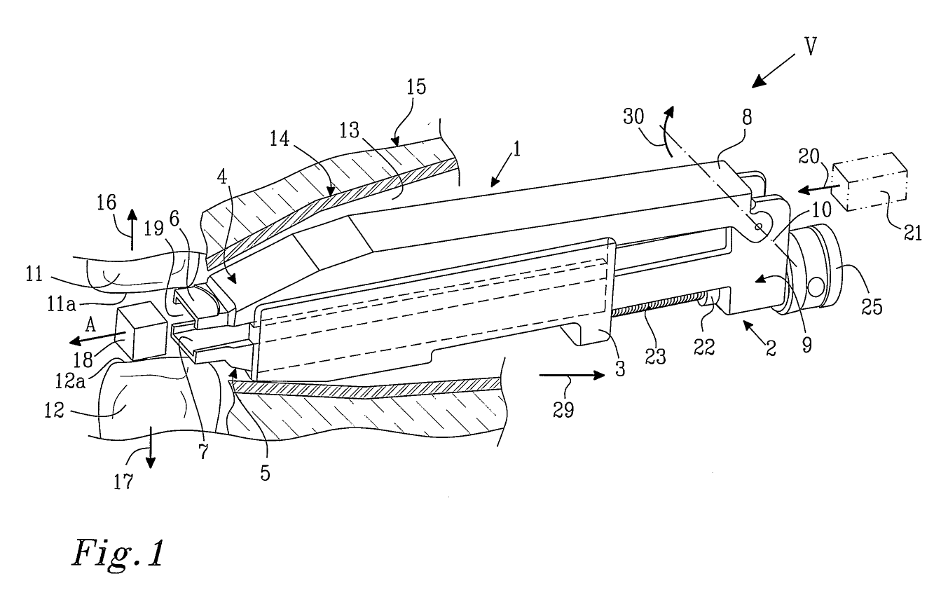

FIG. 1 in a perspective view obliquely from above shows the tool in cooperation with a pair of vertebras via a passage formed by a member expanding and maintaining a surgical operation in an expanded condition,

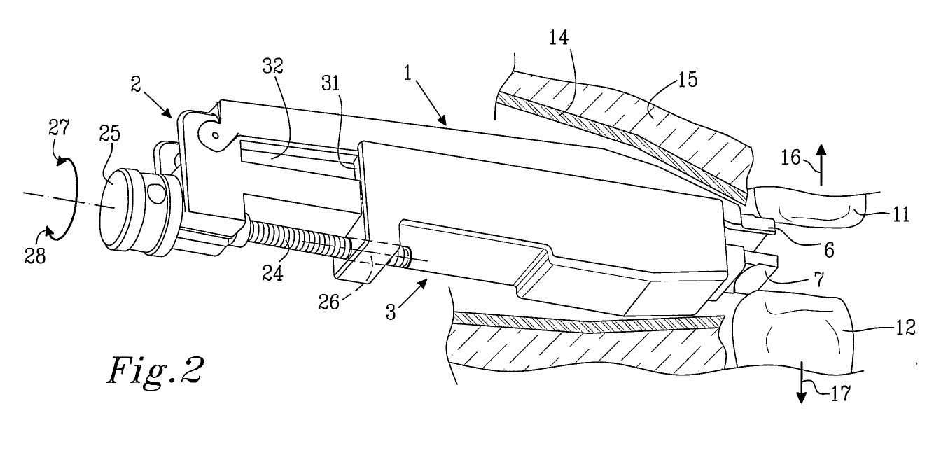

FIG. 2 in a perspective view obliquely from below the tool according to FIG. 1,

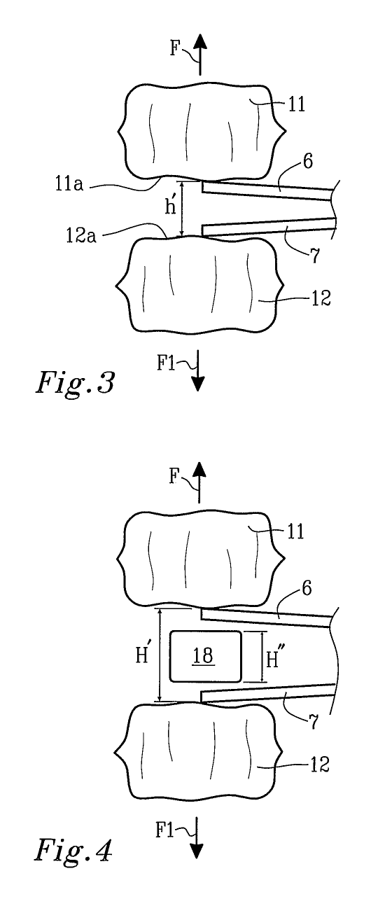

FIG. 3 shows the principle for the attack function of the tool against a pair of vertebras, which shall be separated by means of the tool,

FIG. 4 shows the principle for the functional practice of the tool on the pair of vertebras, which are separated thus, that the material of resilient character is applicable,

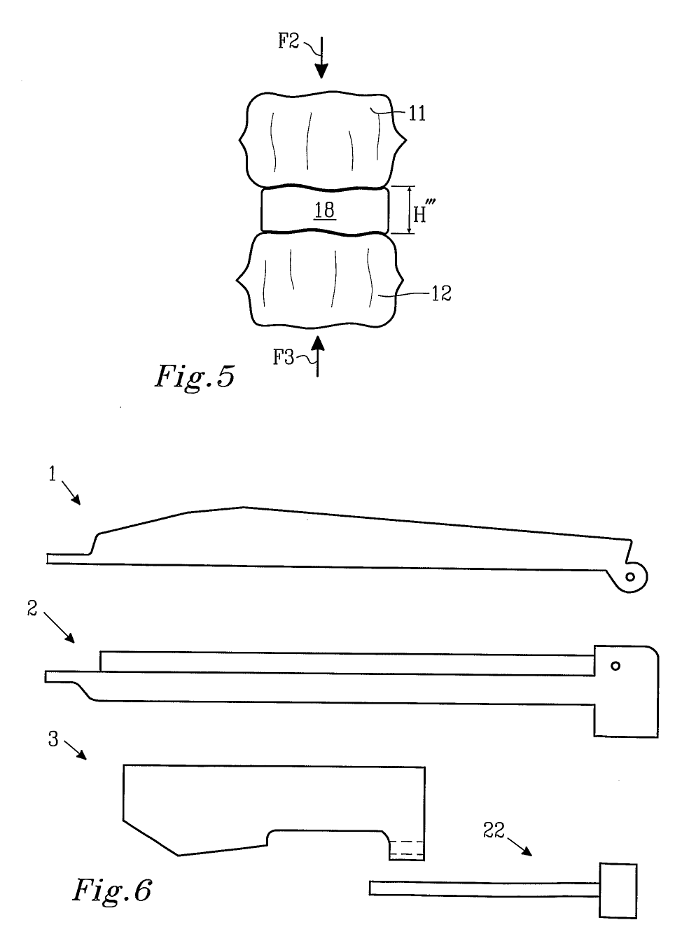

FIG. 5 shows the principle of how the vertebras have sprung back due to the influence of the muscles and begun cooperating with the resilient material,

FIG. 6 shows the principle of the separating function for the tool for giving a possibility of cleaning the different parts of the tool, and

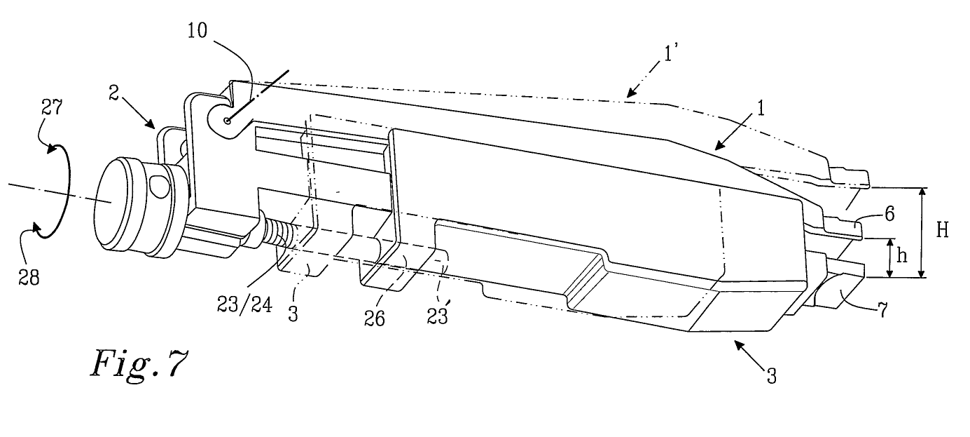

FIG. 7 shows in perspective obliquely from below the tool taking up first and second (dash-lines) positions for the portions.

In FIG. 1 the tool is designated V. The tool incorporates i.a. first and second units 1 and 2, respectively and a third unit 3. The first and second units, at their first ends 4 and 5, support portions 6 and 7. The units 1 and 2 are mutually pivotably supported at their opposite ends 8 and 9. The support points thereby are arranged thus that the distance between the portions 6 and 7 can be expanded and minimized in relation to each other. The pivot axis between the units 1 and 2 is shown at 10. In FIG. 1 two adjacent vertebras are indicated 11 and 12. The portions 6 and 7 are adapted to cooperate with the vertebras 11 and 12. The cooperation is effected via a passage 13 in a member 14 applied in a surgical incision in the human body 15. The surgical incision can be made in a known manner and the member 14 is characterized by a cornet shaped/cone shaped or cylindrical design. Via the passage 13 the portions 6 and 7 can come to engagement against the vertebras 11 and 12 via surfaces 11a and 12a thereon. After this cooperation the pivotal actuation via the axis 10 result in that the units 1 and 2, and therefore the portions 6 and 7 will come to cooperate with the vertebras 11 and 12 for the separation thereof in the direction of the arrows 16 and 17. A distance or a space A is thereby formed between the vertebra surfaces 11a and 12a, which distance results in that a unit 18 is insertable between the vertebras 11 and 12. In the embodiment, the unit 18 can be inserted via a channel 19 formed between the units 1 and 2, and which extends through the tool portions 1 and 2. The unit 18 thereby can be actuated from the ends 8 and 9 of the tool in the direction of the arrow 20. A tool, e.g. a bar 21, is shown in symbolic form in FIG. 1. By means of this tool the unit 18 can be inserted at the unit ends 8, 9 in the channel 19. The tool 1 also has an actuating member 22, which incorporates a screw part 23, which in the figure is shown at 24. In the embodiment, the actuating member is also provided with a handle 25, by means of which the screw can be screwed in and out in an internal thread 26 in the third unit 3. The actuating member is supported in the second unit 2, at the end 9 thereof. The pivoting directions for the handle 25 in the figure are shown at 27 and 28. At actuation from the actuating member 22, i.e. at turning of the handle 25, the third unit 3 is displaced in relation to the second unit 2. At a displacement in the direction of the arrow 29 the third unit 3 is pulled towards the end 9 of the second unit. During this relative displacement the third unit acts upon the first unit in the direction of the arrow 30. This action results in that the portions 6 and 7 are separated from each other in relation to the positions shown in FIG. 1. In a corresponding manner the distances between the portions when the third unit is displaced relative to the second unit in another direction, which is opposed to direction 29. The third unit is provided with an internal flange 31 at each side, i.e. two substantially opposed internal flanges are provided on the third unit. Each flange is guided in a recess 32 provided between the first and the third unit. The flanges 31 and the recesses 32 thereby are arranged thus that the third unit acts upon the first unit for its different pivot positions in relation to the second unit.

FIG. 3 intends to show the principal application of the portions 6 and 7 against two vertebras 11 and 12 situated adjacent each other in a vertebral column. The cooperation between the portions and the vertebras can take place via surfaces 11a and 12a, which can be substantially opposed to each other. After application of the tool 1, 2, 3 against the vertebras, i.e. the portions 6 and 7 are positioned to engage the vertebras, is it possible to actuate the tool units 1, 2 and 3 by means of the actuating member 22, thus that actuation forces F, F1, which are directed from each other can act upon the vertebras.

FIG. 4 shows the phase of the function where the vertebras 11 and 12 by the forces F and F1 have been acted upon to a separation position where the unit 18 of resilient material can be inserted between the vertebras 11 and 12 via the channel 19.

Said separating influence on the vertebras takes place against the action of forces F2 and F3, executed of the muscles around the vertebral column. When the tool 1, 2, 3 is removed from the vertebras and the separating action ceases, the vertebras will be pressed towards each other by means of the forces F2 and F3 with the unit 18 positioned there between, see FIG. 5. The unit 18 thus is arrested in its position.

The tool parts 1, 2, 3 and 22 are mutually demountable as shown in FIG. 6, thus that they can be cleaned separately, e.g. by washing.

In FIG. 2 is shown the initial position according to FIGS. 1 and 2 in continuous lines, whereby the distance between the portions 6 and 7 have been indicated as h. Furthermore is shown in dash lines 1′ an actuation position for the first unit relative to the second unit resulting in a distance H, which exceeds the distance h. The pivoting of the first unit in relation to the second unit takes place in accordance with the above about the axis 10. Different positions have also been shown for the third unit in relation to the second unit in continuous lines as above and dash lines 3′ respectively. Different screwed in positions for the crew of the actuating member have been shown in continuous lines 23 and dash lines 23′. In FIG. 3 is shown an existing initial height between the vertebras as h′. In FIG. 4 is shown the distance H′ between the separated vertebras 11, 12. This distance H′ exceeds the height H″ of the unit 18. In FIG. 5 has been defined a distance H′″, which is somewhat smaller than the distance H″ as the muscles around the vertebral column push back the vertebras 11 and 12 towards each other, see the force directions in FIG. 5. The member 14 with the passage 13 is described in detail in the Swedish patent application filed on the same date and by the same applicants “Anordning för operativt ingrepp eller sårbildning”.

The invention is not limited to the above described embodiment but can be subject to modifications within the scope of the following claims and the inventive idea.

1. Device for application of resilient material between vertebras, characterized therein, that it incorporates first and second units, which support portions adapted to cause separating action on the vertebras via surfaces present thereon, that the separating action by means of the portions can be effected by means of an actuating member at or on one or both of the units or portions respectively, and that insertion of the resilient material is arranged to take place at a separation height exceeding the height of the material.

2. Device as claimed in claim 1, characterized therein,
that it incorporates a third unit, which is longitudinally displaceable relative to the first and second units.

3. Device as claimed in claim 2, characterized therein,
that the actuating member in dependency of actuating movements causes the longitudinal displacement of the third unit in relation to the first and the second units.

4. Device as claimed in claim 2, characterized therein,
that the third unit at its longitudinal displacement directed away from the portions, is adapted to cause separating movement between the first and second units and therefore separating motion between the portions.

5. Device as claimed in claim 4, characterized therein,
that the third unit at its inner side carries a flange which can cooperate with the first unit for causing the separating movement between the first and second units.

6. Device as claimed in claim 2, characterized therein,
that the portions are situated at the first ends of the first and second units, and that the actuating member is arranged at the second ends of the first and second units.

7. Device as claimed in claim 6, characterized therein,
that the first and the second units are pivotably and detachably supported at the second ends of the first and second units.

8. Device as claimed in claim 2, characterized therein,
that the actuating member incorporates a screw, which is supported at one of its ends in a threaded recess in the third unit and a manual actuating member, e.g. a handle, which is arranged to cause in dependency of a pivoting motion the longitudinal displacement of the third unit in relation to the second unit.

9. Device as claimed in claim 2, characterized therein,
that the first and second units are arranged to form an internal channel, via which channel the resilient material is insertable between the separated vertebras.

10. Device as claimed in claim 2, characterized therein,
that it via the portions is insertable via a passage provided between the external side of the human body and the vertebras by means of a member arranged to expand and maintain a surgical operation in an expanded condition.

11. Device as claimed in claim 10, characterized therein,
that the last mentioned member incorporates a spiral, e.g. made from plastic material, and a cloth positioned outside on the spiral and arranged to prevent intrusion of solid body products in the passage.

12. Device as claimed in claim 11, characterized therein,
that the member or the spiral with the cloth substantially is shaped as a cone or a frustrum of a cone with the smaller part facing the vertebras.

13. Device as claimed in claim 2,
characterized therein,
that the first, second and third units are demountable for permitting cleaning thereof.

14. Device as claimed in claim 3,
characterized therein,
that the third unit at its longitudinal displacement directed away from the portions, is adapted to cause separating movement between the first and second units and therefore separating motion between the portions.
